(19) United States
(12) Patent Application Publication
(10) Pub. No.: US 2021/0154327 A1
(43) Pub. Date: May 27, 2021

(54) PROTRANSDUZIN-D - IMPROVED ENHANCER OF GENE TRANSFER

(71) Applicant: Pharis Biotec GmbH, Hanover (DE)

(72) Inventors: Wolf-Georg FORSSMANN, Hanover (DE); Rudolf RICHTER, Kömigstein (DE)

(21) Appl. No.: 16/641,841

(22) PCT Filed: Aug. 29, 2018
(86) PCT No.: PCT/EP2018/073194

§ 371 Date: Mar. 5, 2020

(30) Foreign Application Priority Data
Aug. 29, 2017 (EP) 17188384.6

(51) Int. Cl. A61K 48/00

(52) U.S. Cl. CPC A61K 48/0041; A61K 48/005

(57) ABSTRACT

The invention relates to a polypeptide having the sequence Z¹-Gln-Ala-Lys-Ile-Lys-Gln-Ile-Ile-Asn-Met-Trp-Gln-Z². The polypeptide is used for retroviral transfection/transduction.

The present application relates to an improved enhancer of gene transfer, namely protransduzin D (PTD-D), an improvement over the transduction enhancers PTD-A and PTD-B, to its polypeptide, to an N-terminally protected polypeptide, to a medicament containing said polypeptide, to said polypeptide for use in gene therapy, to a method for enhancing the infection of a cell by a genetically engineered viral construct, and to the use of said polypeptide for amplification for transfection or transduction.

The introduction of genetic material for changing specific cell functions has become an indispensable tool of biological-medical basic and applied research since the cloning of the first human genes and recombinant production. There is a continuous progress in the methods of gene introduction, which leads to optimization of gene transfer.

Studies for the development of transduction enhancers led to the discovery of protransduzin-A, a peptide from a viral envelope protein of HIV, which surprisingly was suitable for improving the lentiviral transduction of gene material into the nucleus above an unprecedented extent (Yolamanova et al., Nature Nanotechnology). Thus, it could be shown, for example, that HIVs preincubated with different concentrations (1-100 ug/ml) of protransduzin A (synonym: EF-C) exhibit an infection rate with reporter cells that is increased by several powers of ten as compared with the gold standard “retronectin”. As the mechanism of action, it was assumed that EF-C forms fibrillary structures that are capable of binding and concentrating viruses and accordingly amplifying the entry of the viruses into the cell. In addition to the infection with viral particles, EF-C enhances the transduction of lentiviral and retroviral particles with high efficiency in a wide variety of human cell types (T cells, glial cells, fibroblasts, hematopoietic stem cells) applied in gene therapy (Jan Munch et al., Nature Nanotechnology, Vol. 8, No. 2, pp. 130-136). EP 2 452 947 A1 also relates to protransduzin A.

Cysteine belongs to the group of polar neutral amino acids. Further, because of its unique functional thiol group, cysteine is an amino acid whose modification or even replacement by another amino acid, especially from a different group of amino acids, is never considered. Like Cys, the amino acids Tyr, Asp, Ser, Gly, Gln, Thr are part of the group of polar, but neutral amino acids. However, the skilled person would shy away from replacement by amino acids from other groups, because the effects of such replacement on the secondary structure of the peptide modified by exchange are difficult to foresee. Thus, the skilled person would not consider replacing of Cys of protransduzin by amino acids from the group of non-polar hydrophobic amino acids.

However, it has now surprisingly been found that the amino acid Cys in protransduzin may be replaced by alanine without leading to a significant change of the effectiveness of the modified polypeptide as a transduction enhancer.

According to the invention, a modified protransduzin is provided, which on the one hand has an effectiveness comparable to that of PTD-A as a transduction enhancer, has a higher storage stability and ensures a simpler handling in the application thereof as a transduction enhancer, which is an enormous advantage in GMP production.

The polypeptide according to the invention has at least 80% or 90% sequence identity, especially 95% sequence identity, with the sequence

Z¹-Gln-Ala-Lys-Ile-Lys-Gln-Ile-Ile-Asn-Met-Trp-Gln-
Z².

Z¹ are independently of one another the N-terminal end of the polypeptide, or independently of one another the amino acids Leu or Ser, or the following peptides:

### Ser-Asn, Ser-Asn-Asn, Ser-Asn-Asn-Ile, Ser-Asn-Asn-Ile-Thr, Thr-Leu, Ile-Thr-Leu, Asn-Ile-Thr-Leu, Asn-Asn-Ile-Thr-Leu, or Ser-Asn-Asn-Ile-Thr-Leu,

Z² are independently of one another the C-terminal end of the polypeptide, or independently of one another the amino acids Gly or Glu, or the following peptides:

Glu-Val,
Glu-Val-Gly,
Glu-Val-Gly-Lys,
Glu-Val-Gly-Lys-Ala,
Glu-Val-Gly-Lys-Ala-Met,
Glu-Val-Gly-Lys-Ala-Met-Tyr,
Glu-Val-Gly-Lys-Ala-Met-Tyr-Ala,
Glu-Val-Gly-Lys-Ala-Met-Tyr-Ala-Pro,
Glu-Val-Gly-Lys-Ala-Met-Tyr-Ala-Pro-Pro,
Glu-Val-Gly-Lys-Ala-Met-Tyr-Ala-Pro-Pro-Ile,
Glu-Val-Gly-Lys-Ala-Met-Tyr-Ala-Pro-Pro-Ile-Glu,
Glu-Val-Gly-Lys-Ala-Met-Tyr-Ala-Pro-Pro-Ile-Glu-
Gly,
Glu-Gly,
Ile-Glu-Gly,
Pro-Ile-Glu-Gly,
Pro-Pro-Ile-Glu-Gly,
Ala-Pro-Pro-Ile-Glu-Gly,
Tyr-Ala-Pro-Pro-Ile-Glu-Gly,
Met-Tyr-Ala-Pro-Pro-Ile-Glu-Gly,
Ala-Met-Tyr-Ala-Pro-Pro-Ile-Glu-Gly,
Lys-Ala-Met-Tyr-Ala-Pro-Pro-Ile-Glu-Gly,
Gly-Lys-Ala-Met-Tyr-Ala-Pro-Pro-Ile-Glu-Gly,
Val-Gly-Lys-Ala-Met-Tyr-Ala-Pro-Pro-Ile-Glu-Gly,
or
Glu-Val-Gly-Lys-Ala-Met-Tyr-Ala-Pro-Pro-Ile-Glu-
Gly.

The improvement that can be achieved by the polypeptide according to the invention involves an increased stability of the transduction enhancer, which may be employed for therapeutic use. An efficient gene transduction in cells for therapeutical application can reduce the required amount of viral particles used for gene transduction, for example. Further, the number of infection cycles necessary for an efficient transduction can be reduced. By using the polypeptide according to the invention as a transduction enhancer, the duration of in-vitro culturing for proliferating the gene-modified cells and the amount of cells to be removed from a patient (e.g., by leukapheresis) can be reduced, and in some cases, an efficient and non-toxic in-vivo gene transduction may be enabled by reducing the virus load in vivo. Further, the quick handling of an efficient transduction enhancer reduces the load on the cells to be transduced.

Because of its improved stability, the polypeptide according to the invention allows for a better prediction of the effectiveness of the transduction enhancer even after prolonged storage of the substance.

Because of its higher stability, the polypeptide according to the invention also allows for the production of larger peptide batches that can be stored longer, as compared to PTD-A. The lesser reactivity of the polypeptide according to the invention as compared to PTD-A leads to its having a lower cytotoxic activity. In transduction, this leads to a higher yield of transduced cells. In particular, the invention relates to the polypeptide having at least 80% sequence identity, especially 90% sequence identity, with the sequence

Gln-Ala-Lys-Ile-Lys-Gln-Ile-Ile-Asn-Met-Trp-Gln.

According to the invention, the term “polypeptide according to the invention” also includes those related polypeptides that are formed by varying the amino acids in the polypeptide chain of the polypeptide according to the invention, but still have a comparable and sufficient effectiveness, which can be determined, for example, in the following bioassay.
The transduction efficiency can be tested, for example, by using primary activated CD4+/CD8+ enriched T cells, which are cultured with CD3/CD28 beads for 3 days as target cells, and lentiviral and retroviral vectors encoding Green Fluorescent Protein (GFP). PTD-D can be tested, for example, against PTD-A and retronectin as the gold standard of transduction enhancers.

PTD-A and PTD-D are employed in an assay at a concentration of, for example, 25 μg/ml. The target cells are employed at a concentration of, in particular, 10³ to 10⁶ cells/ml. The mix is incubated for 8 to 16 hours, and subsequently the cells are washed. Then, the cells are cultured, for example, for another 4 days. Then, on day 7, the proportion of GFP+ T cells is determined by means of flow centrifugation, and the cell count and vitality are determined.

In particular, a homologous peptide is a polypeptide related to the sequence of the polypeptide according to the invention, in which replacements or deletions of amino acids were performed to the extent mentioned. In particular, exchanges of amino acids having similar properties, for example, similar polarities, are possible. Thus, exchanges of arginine and lysine, glutamic acid and aspartic acid, glutamine, asparagine and threonine, glycine, alanine and proline, leucine, isoleucine and valine, thyrosine, phenylalanine and tryptophan as well as serine and threonine, are widespread.

At position 1 of the sequence, there is preferably the amino acid glutamine. At positions 3 and 5, there are preferably basic amino acids, preferably lysine. At positions 1, 4, 6, 7, 8, 9, 10, 11, and 12, there are mostly neutral amino acids. The sequence may be extended or truncated N-terminally and/or C-terminally. The sequence of the monomer may be N-terminally extended by C-terminal parts or the whole amino acid sequence NH₂-Ser-Asn-Asn-Ile-Thr-Leu-COOH.

The sequence of the monomer may be C-terminally extended by N-terminal parts or the whole amino acid sequence NH₂-Glu-Val-Gly-Lys-Ala-Met-Tyr-Ala-Pro-Pro-Ile-Glu-Gly-COOH. The polypeptides according to the invention share the common property of forming insoluble aggregates in aqueous solutions. The monomers consist of 4 to 25 amino acids, preferably of 10 to 20 amino acids.

Further, the homologous molecules share the common property of forming insoluble aggregates in aqueous solutions, and enhancing the transduction of target cells with lentiviral or retroviral vectors.

In one embodiment of the invention, the N-terminal end of the amino acid chain constituting the polypeptide according to the invention is modified with a chemical group selected from the group consisting of one or two alkyl groups, such as methyl, ethyl, propyl or butyl groups, an acyl group, such as an acetyl or propionyl group, or the amino acid pyroglutamic acid,

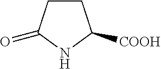

which forms the N-terminal end.
The invention also relates to a medicament containing at least one polypeptide according to the invention.
The invention also relates to a polypeptide for use in gene therapy for treating diseases that are treatable with gene therapy.

The present invention also relates to a method for enhancing the infection of a cell by a virus, comprising the steps:

providing the polypeptide according to claim 1 or 2 dissolved in an organic solvent;

adding the polypeptide to an aqueous solution to form insoluble aggregates of the polypeptide;

mixing the solution from the last preceding step; and

culturing the cells in the presence of at least one polypeptide according to the invention.

The present invention also relates to the use of at least one polypeptide according to the invention for enhancing the infection of a cell with a virus.

The present invention also relates to a kit containing at least one polypeptide according to the invention.

The peptide according to the invention can be prepared, for example, by the method according to Merrifield with Fmoc-protected amino acids.

This method works with Fmoc-protected derivatives, i.e., with (9-fluorenylmethoxycarbonyl)-protected amino acids, in a stepwise solid phase synthesis according to the Merrifield principle, especially on a Wang resin preloaded with Fmoc-L-glutamine (0.59 mmol/g, 100-200 mesh) as a solid support on the synthesizer ABI-433.

The activation of the Fmoc-L-amino acids, which were typically employed in a tenfold molar excess, is performed with R2-(1H-benzotriazol-1-yl)-1,1,3,3-tetramethyluronium hexafluorophosphatel (HBTU, 100 mmol/1) with additions of 0.5 M 1-hydroxybenzotriazole (HOBt) and 2 M diisopropylethylamine (DIEA) in N-methyl-2-pyrrolidinone (NMP) at room temperature.

The individual acylation reactions take 45 minutes, and the Fmoc deprotection with 20% piperidine takes 15 minutes.

The following amino acid derivatives and related orthogonal acid-cleavable side chain protective groups are employed for synthesis:

### Fmoc-L-Asn(Trt), Fmoc-L-Ala(Trt), L-pGlu, Fmoc-L-Gln(Trt), Fmoc-L-Ile, Fmoc-L-Lys(Boc), Fmoc-L-Met and Fmoc-L-Trp(Boc).

After cleaving the resin support from the peptidyl resin with 94% trifluoroacetic acid (TFA), 3% ethanedithiol (EDT) and 3% demineralized water, the raw peptide is precipitated in cold tert-butyl methyl ether, the raw peptide is centrifuged off as a pellet, and the supernatant is discarded.

The subsequent chromatographic purification of the raw peptide is effected in a preparative way by gradient elution.

The difference between protransduzin A according to EP 2 452 947 A1 and protransduzin D resides in the fact that protransduzin D is Cys2/alanine-substituted according to the invention. The difference between protransduzin B according to WO 2014/177635 A1 and protransduzin D resides in the fact that protransduzin D is Cys2/alanine-substituted according to the invention, and that the synthetic L-pyroglutamic acid (pGlu) is inserted N-terminally in exchange for synthetic L-glutamine (Gln). The original glutamine is modified by ring closure to form a lactam.

### EXAMPLE

The transduction efficiency was examined on activated CD3+ T cells. Five different PTD batches were tested against different controls and against retronectin.

For transduction, cryopreserved CD4+/CD8+ enriched T cells were used. These were thawed, and cultured with CD3/CD28 beads for 3 days. After removing the beads after 3 days, the T cells were transduced with a GFP vector, which was obtained by means of the producer cell line HG820#4E912#4.3. Two different MOIs were used. The cells were then cultured for another 4 days. On day 7, the cell count and the vitality of the cells were determined using a Nucleo counter NC-200. The proportion of GFP+ T cells was determined by means of flow centrifugation.

Prestimulation, Removing the Beads, Transduction, Expansion

The CD4+/CD8+ T cells were thawed on day 0 using a Barkey Plasmatherm device, and washed once with 1×PBS. The cells were resuspended with the addition of cell culture medium X-vivo 15+2 mM Glutamax+5% CTS “Immune Cell Serum Replacement” with adding 450 IU/ml IL-7 and 50 IU/ml IL-15 at a density of 1×10⁶ viable cells/ml. For activating the cells, 3 (three) CD3/CD28 Dynabeads per cell were added, and cultured until day 3.

On day 3, the beads were removed with a MixMate from Eppendorf and a MaxSep magnet from Baxter. After concentration by centrifugation, the cells were transduced with a GFP vector at a concentration of MOI 2 or MOI 4. For the controls, transduction with retronectin and with a virus and without an addition was performed. A non-transduced control was used as a negative control.

The different protransduzin peptides were dissolved in DMSO to 10 mg/ml (stock solution). The stock solution was diluted with 1×PBS to a concentration of 1 mg/ml, and incubated for at least 10 min, in order that PTD fibrils can form. The viral supernatant was mixed with the Medium and Serum Replacement to obtain the necessary concentration. Subsequently, the different PTDs were added, to obtain a concentration of 50 μg/ml. The mixture was incubated at room temperature for a minimum of 5 min. After the incubation, the cells were added, so that a cell density of 5×10⁵ cells/ml and a final PTD concentration of 25 μl/ml were obtained. The cells were incubated in 6-well plates at 37° C., 5% CO₂ and under a high humidity for 16+/−4 hours to day 4.

On day 4, the cell count and viability were determined, which yields improved results in the PTDs as compared to previous analogues. The cells were transferred into T-25 culture bottles with 5 ml of cell culture medium, and incubated at 37° C., 5% CO₂ and under a high humidity.

On day 5, 10 ml of fresh cell culture medium was added.
On day 7, the volume of the culture medium of each culture bottle was determined. Subsequently, the cell count, the viability and the transduction rate were determined by FACS analysis.

### Determining the Cell Count and the Viability

The cell count and the viability were determined by means of a NucleoCounter NC-200 using disposable Vial cassettes.

### Determining the GFP Expression by Flow Cytometry

The number of GFP+ T cells (CD3+) was determined by means of a FACSVerse Flow Cytometer. For measurement, 1.5×10⁶ viable cells were washed with 1×PBS. After centrifugation, the cell pellet was taken up in 300 μl 1×PBS and 3 μl FVS780 solution. Per test, two FACS tubes were stained:
Tube 1: GFP/FVS 780/BV421-unstained (FMO control for CD3-BV421)
Tube 2: GFP/FVS 780/CD3-BV421 [5 μl]
For each tube, 1×10⁶ cells were added, and incubated at RT in the dark for 15 min After incubation, the cells were admixed with staining buffer BSA, and centrifuged. Subsequently, the cells are taken up in 350 μl staining buffer BSA, and analyzed by flow cytometry within 180 min For the calculation of the absolute number of GFP+ cells in a T-25 culture bottle, the results were corrected against the non-transduced control.

1. A polypeptide having at least 80% or 90% sequence identity, especially 95% sequence identity, with the sequence
Z¹-Gln-Ala-Lys-Ile-Lys-Gln-Ile-Ile-Asn-Met-Trp-Gln-
Z².
wherein
Z¹ represents the N-terminal end of the polypeptide, or independently of one another are the amino acids Leu or Ser, or the following peptides:
Ser-Asn, Ser-Asn-Asn, Ser-Asn-Asn-Ile, Ser-Asn-Asn-Ile-Thr, Thr-Leu, Ile Thr Leu, Asn-Ile-Thr-Leu, Asn-Asn-Ile-Thr-Leu, or Ser-Asn-Asn-Ile-Thr-Leu,
Z² represents the C-terminal end of the polypeptide, or independently of one another are the amino acids Gly or Glu, or the following peptides:
Glu-Val,
Glu-Val-Gly,
Glu-Val-Gly-Lys,
Glu-Val-Gly-Lys-Ala,
Glu-Val-Gly-Lys-Ala-Met,
Glu-Val-Gly-Lys-Ala-Met-Tyr,
Glu-Val-Gly-Lys-Ala-Met-Tyr-Ala,
Glu-Val-Gly-Lys-Ala-Met-Tyr-Ala-Pro,
Glu-Val-Gly-Lys-Ala-Met-Tyr-Ala-Pro-Pro,
Glu-Val-Gly-Lys-Ala-Met-Tyr-Ala-Pro-Pro-Ile,
Glu-Val-Gly-Lys-Ala-Met-Tyr-Ala-Pro-Pro-Ile-Glu,
Glu-Val-Gly-Lys-Ala-Met-Tyr-Ala-Pro-Pro-Ile-Glu-
Gly,
Glu-Gly,
Ile-Glu-Gly,
Pro-Ile-Glu-Gly,
Pro-Pro-Ile-Glu-Gly,
Ala-Pro-Pro-Ile-Glu-Gly,
Tyr-Ala-Pro-Pro-Ile-Glu-Gly,
Met-Tyr-Ala-Pro-Pro-Ile-Glu-Gly,
Ala-Met-Tyr-Ala-Pro-Pro-Ile-Glu-Gly,
Lys-Ala-Met-Tyr-Ala-Pro-Pro-Ile-Glu-Gly,
Gly-Lys-Ala-Met-Tyr-Ala-Pro-Pro-Ile-Glu-Gly,
Val-Gly-Lys-Ala-Met-Tyr-Ala-Pro-Pro-Ile-Glu-Gly,
or
Glu-Val-Gly-Lys-Ala-Met-Tyr-Ala-Pro-Pro-Ile-Glu-
Gly.

2. The polypeptide according to claim 1, having at least 90% sequence identity, especially 95% homology, with the sequence
Gln-Ala-Lys-Ile-Lys-Gln-Ile-Ile-Asn-Met-Trp-Gln.

3. The polypeptide according to claim 1, wherein the N-terminal end is modified with a chemical group selected from the group consisting of one or two alkyl groups, such as methyl, ethyl, propyl or butyl groups, an acyl group, such as an acetyl or propionyl group, or the amino acid pyroglutamic acid forms the N-terminal end.

4. A medicament containing a polypeptide according to claim 1.

5. A polypeptide according to claim 1 for use in gene therapy for treating diseases that are treatable with gene therapy.

6. A method for enhancing the infection of a cell by a virus, comprising the steps:
providing the polypeptide according to claim 1 dissolved in an organic solvent;
adding the polypeptide to an aqueous solution to form insoluble aggregates of the polypeptide;
mixing the solution from the last preceding step; and
culturing the cells in the presence of the polypeptide according to claim 1.

7. Use of the polypeptide according to claim 1 for enhancing the infection of a cell by a virus.

8. A kit containing a polypeptide according to claim 1.


SEQUENCE LISTING (26 sequences)

SEQ ID NO: 1 - protein, 12 aa, human
QAKIKQIINMWQ

SEQ ID NO: 2 - protein, 4 aa, human
SNNI

SEQ ID NO: 3 - protein, 5 aa, human
SNNIT

SEQ ID NO: 4 - protein, 4 aa, human
NITL

SEQ ID NO: 5 - protein, 5 aa, human
NNITL

SEQ ID NO: 6 - protein, 6 aa, human
SNNITL

SEQ ID NO: 7 - protein, 4 aa, human
EVGK

SEQ ID NO: 8 - protein, 5 aa, human
EVGKA

SEQ ID NO: 9 - protein, 6 aa, human
EVGKAM

SEQ ID NO: 10 - protein, 7 aa, human
EVGKAMY

SEQ ID NO: 11 - protein, 8 aa, human
EVGKAMYA

SEQ ID NO: 12 - protein, 9 aa, human
EVGKAMYAP

SEQ ID NO: 13 - protein, 10 aa, human
EVGKAMYAPP

SEQ ID NO: 14 - protein, 11 aa, human
EVGKAMYAPPI

SEQ ID NO: 15 - protein, 12 aa, human
EVGKAMYAPPIE

SEQ ID NO: 16 - protein, 13 aa, human
EVGKAMYAPPIEG

SEQ ID NO: 17 - protein, 4 aa, human
PIEG

SEQ ID NO: 18 - protein, 5 aa, human
PPIEG

SEQ ID NO: 19 - protein, 6 aa, human
APPIEG

SEQ ID NO: 20 - protein, 7 aa, human
YAPPIEG

SEQ ID NO: 21 - protein, 8 aa, human
MYAPPIEG

SEQ ID NO: 22 - protein, 9 aa, human
AMYAPPIEG

SEQ ID NO: 23 - protein, 10 aa, human
KAMYAPPIEG

SEQ ID NO: 24 - protein, 11 aa, human
GKAMYAPPIEG

SEQ ID NO: 25 - protein, 12 aa, human
VGKAMYAPPIEG

SEQ ID NO: 26 - protein, 13 aa, human
EVGKAMYAPPIEG